(19) United States
(12) Patent Application Publication
(10) Pub. No.: US 2016/0008168 A1
(43) Pub. Date: Jan. 14, 2016

(54) DEVICE AND METHOD FOR PORTABLE THERMAL SUPPORT

(71) Applicant: General Electric Company, Schenectady, NY (US)

(72) Inventors: Karen Starr, Monkton, MD (US); Steven Falk, Baltimore, MD (US); Harry Belsinger, JR., Forest Hill, MD (US)

(21) Appl. No.: 14/329,056

(22) Filed: Jul. 11, 2014

(51) Int. Cl. A61F 7/08; A47D 13/02

(52) U.S. Cl. CPC A61F 7/08; A47D 13/02

(57) ABSTRACT

The present application discloses a transfer device for warming an infant patient during movement of the patient. The transfer device comprises a center section positionable beneath the patient, and the center section comprises a warming device. The transfer device also comprises a first side section connected to the center section and including a first handle and a second side section connected to the center section and including a second handle. The first and the second side sections are movable toward each other such that the first and second handles are located proximate to each other and the first and second side sections surround the patient.

### BACKGROUND OF THE INVENTION

The subject matter disclosed herein relates to warming an infant. More specifically, the present disclosure relates to an infant transfer device (sling) that can be used to provide thermal and physical support during movement while providing minimal patient stimulation.

Newborn infants require thermal support immediately after birth to assist with convective, evaporative, conductive and radiant heat loss. Without thermal support from skin to skin contact, drying or heating devices, newborns, especially preterm newborns, are likely to become hypothermic. Hypothermia is a leading cause of neonatal morbidities and mortalities. Current methods for preventing heat loss include swaddling with a blanket or a food grade plastic bag, and positioning the infant on a warming mattress before introducing the infant to the next level of care in an incubator or warmer.

Infant patients can also be extremely sensitive to physical touch. For example, the current standard practice used to transfer an infant patient is for a nurse or other caregiver to carefully slide a hand (or two) under the infant patient and manually lift the patient. This physical contact can cause patient stimulation which in high risk patients can introduce unwanted and harmful stress to the infant patient.

Therefore, a device capable of providing thermal and physical support is desired to aid the newborn infant in the transition from intrauterine to extrauterine life.

### BRIEF DESCRIPTION OF THE INVENTION

The above-mentioned shortcomings, disadvantages and problems are addressed herein which will be understood by reading and understanding the following specification.

In an embodiment, a transfer device for warming an infant patient during movement of the patient is provided comprising a center section positionable beneath the patient, the center section comprising a warming device. The transfer device also comprises a first side section connected to the center section and including a first handle and a second side section connected to the center section and including a second handle. The first and the second side sections are movable toward each other such that the first and second handles are located proximate to each other and the first and second side sections surround the patient.

In another embodiment, a transfer device for warming an infant patient during movement of the patient is provided, comprising a center section positionable beneath the patient. The transfer device also comprises a warming device removably positioned within the center section and a first side section connected to the center section. The transfer device further comprises a second side section connected to the center section. The first and second side sections are sized to surround the infant patient when the first and second side sections are brought towards each other.

In another embodiment, a method of warming an infant patient during movement of the patient is provided, comprising activating a warming device and inserting the warming device into a center section of a patient transfer device. The method also comprises positioning the transfer device beneath the patient, the transfer device comprising a first side section and a second side section, the first side section and the second side section connected to the center section, and wherein the infant is positioned on the center section. The method further comprises lifting first and second sections to surround the infant patient.

Various other features, objects, and advantages of the invention will be made apparent to those skilled in the art from the accompanying drawings and detailed description thereof.

### BRIEF DESCRIPTION OF THE DRAWINGS

FIG. 1 is an interior perspective view of a first embodiment of a patient transfer device of the present disclosure;

FIG. 2 is an exterior perspective view of an embodiment of the patient transfer device;

FIG. 3 is a view showing the transport of an infant patient using an embodiment of the patient transfer device of the present disclosure;

FIG. 4 is a front perspective view of an embodiment of the patient transfer device;

FIG. 5 is a front perspective view of an embodiment of the patient transfer device;

FIG. 6 is a view showing the transport of an infant patient using an embodiment of the patient transfer device of the present disclosure; and

FIG. 7 is flowchart of a method of warming an infant patient in accordance with an embodiment of the present disclosure.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

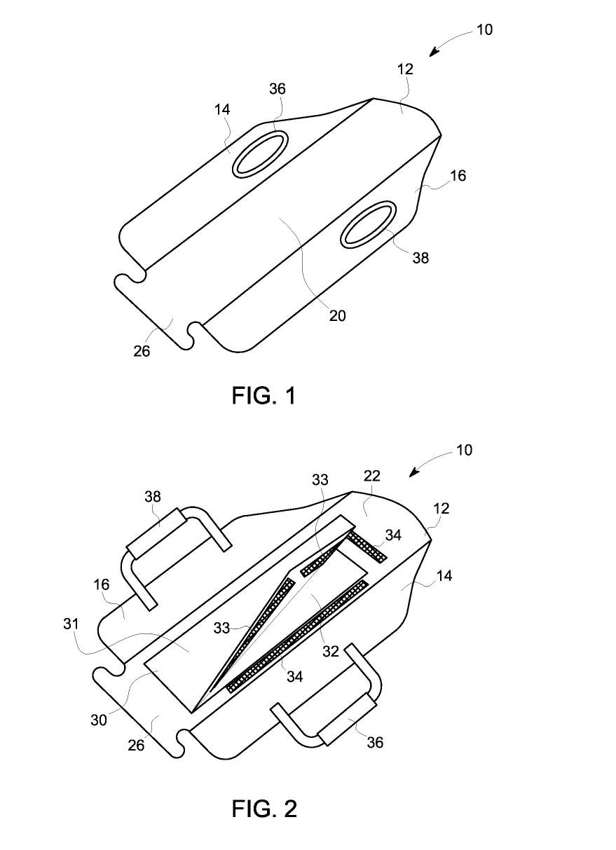

FIG. 1 illustrates an embodiment of a patient transfer device 10 of the present disclosure. The patient transfer device 10 can be used to handle an infant patient, such as a newborn or neonate, and to transfer the infant patient from one location to another while minimizing physical contact between a physician or caregiver and the patient while still providing thermal and physical support to the infant patient.

The patient transfer device 10 comprises a center section 12, a first side section 14 connected to the center section 12, and a second side 16 connected to the center section 12. In the depicted embodiment, the second side section 16 is connected to the center section 12 opposite the first side section 14, but other configurations of the first and second sides 14, 16 with respect to the center section 12 may be envisioned. In some embodiments, such as that depicted in FIG. 1, the patient transfer device 10 may also comprise an end portion 26. However, the end portion 26 could be eliminated while also operating within the scope of the present disclosure.

The patient transfer device 10 has an interior side 20, which is adapted to receive and be in general contact with an infant patient (as depicted in FIG. 1), and an opposing exterior side 22 (as depicted in FIG. 2).

The first side section 14 includes a handle 36 and the second side section 16 includes a handle 38. In the embodiment depicted in FIG. 1, handles 36, 38 are formed in the first and second side sections 14, 16. However, it should be appreciated that other embodiments of handles 36, 38 are envisioned. For example, as depicted in FIG. 2, the handles 36, 38 may be connected to, but spaced apart from, the first and second side sections 14, 16. In another embodiment (not shown), the handles 36, 38 could be connected to the center section 12 and sized so that the handles 36, 38 could be brought together about first and second side sections 14, 16.

In FIG. 2, the exterior side 22 of the patient transfer device 10 in accordance with an embodiment is shown. The patient transfer device 10 depicted in FIG. 2 is generally similar to that depicted in FIG. 1.

The patient transfer device 10 may comprise a warming device 32. In one embodiment, the warming device 32 is an exothermic chemical reaction pouch. As is commonly known in the art, the pouch may be physically bent, cracked or shaken to mix the components and begin an exothermic, heat-producing reaction. Similar pouches are available commercially as disposable hand warmers. In another embodiment, the warming device 32 is a phase change material. The phase change material, such as a wax, is one that once melted, slowly releases heat until the liquid has solidified, thus changing phase. To warm an infant patient, for example, a phase change material that changes from liquid to solid near 37° C., or within a few degrees in either direction, is desired. In yet another embodiment, the warming device 32 is a battery-operated electrical heating system device that produces thermal support when desired by activating or turning the device on. It should be appreciated that other embodiments of the warming device 32 may be envisioned, and may be any device suitable for providing short term thermal support.

The warming device 32 may be removably positioned within or coupled to the patient transfer device 10. When the warming device 32 is positioned within or coupled to the patient transfer device 10, it is capable of providing conductive thermal support to the infant patient. In the embodiment depicted in FIG. 2, the warming device 32 is coupled to the patient transfer device 10 via a pocket 30. The pocket 30 may be formed, for example, on the exterior side 22 of the patient transfer device 10 between an outer component 31 and the center section 12. Other embodiments of the pocket 30 are envisioned. For example, the pocket 30 may formed on the interior side 20 of the patient transfer device 10 (not shown).

In the embodiment depicted in FIG. 2, the pocket 30 is rectangular in geometry and is fixedly attached on two sides and releasably coupled on the remaining two sides. It should be appreciated that other attachment configurations and geometries may be envisioned. For example, the pocket 30 may be fixedly attached on three sides but open, and not attached or releasably coupled, on the fourth side. In another embodiment, the pocket 30 may not be fixedly attached at all, and instead the pocket 30 may be completely releasably coupled to the center section 12. In yet another embodiment, the pocket 30 may be circular or elliptical and may be fixedly attached along a portion of that geometry.

As depicted in FIG. 2, the pocket 30 is coupled by fasteners 33, 34. Fasteners 33, 34 are opposite portions of a hook and loop fastener, such as Velcro ®. The physical engagement between fasteners 33, 34 allows the pocket 30 to be coupled to the exterior side 22 of the center section 12. Although a hook and loop fastener are shown in this embodiment, it should be understood that different types of fasteners could be utilized while operating within the scope of the present disclosure.

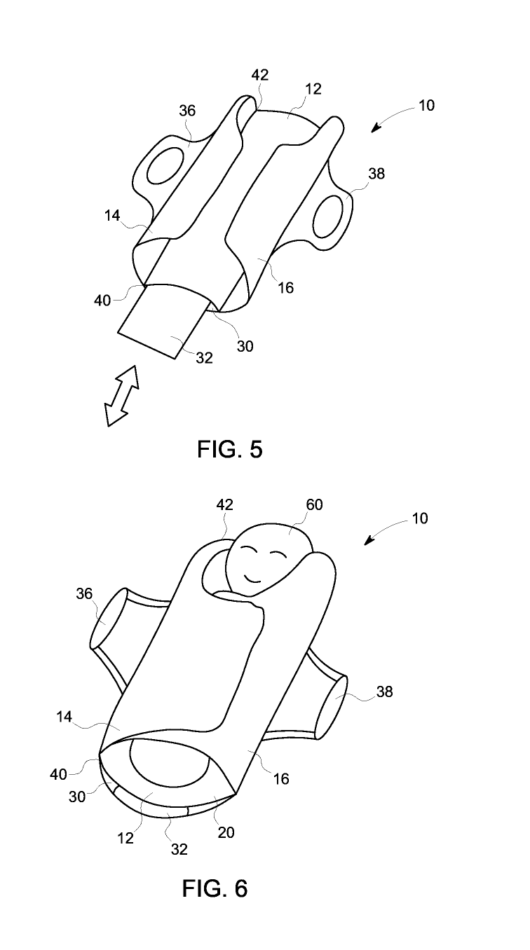

In another embodiment, depicted in FIGS. 4 and 5, the pocket 30 may be formed within the center section 12. The pocket 30 may have an opening at either a proximal end 40 or a distal end 42 of the patient transfer device 10. The warming device 32 may be positioned in the patient transfer device 10 by removably inserting the warming device 32 into the pocket 30 at the proximal end 40 (as is depicted in FIG. 5), or at the distal end 42 (as is depicted in FIG. 4). In another embodiment, the pocket 30 may have more of a sleeve-like configuration with openings at both proximate and distal ends 40, 42. In this embodiment, the warming device 32 may be removably inserted into the pocket 30 at either the proximal end 40 or the distal end 42, or a combination thereof.

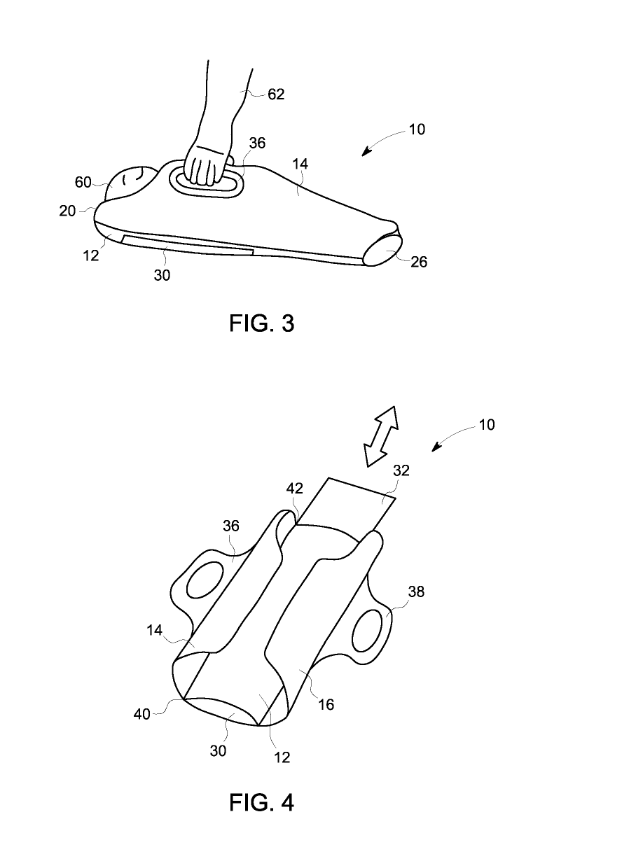

Having described exemplary components of the patient transfer device 10, the operation of the patient transfer device 10 will now be described in accordance with an embodiment. In the embodiment depicted in FIG. 3, an infant patient 60 can be placed on the interior side 20 of the center section 12 and the end portion 26 can be brought up to cover the infant patient's feet while the first and second side sections 14, 16 can be brought towards one another to surround the infant patient 60. The warming device may be inserted into pocket 30 to provide thermal support. When the infant patient 60 is supported by the patient transfer device 10, and the first and second side sections 14, 16 are in proximity to one another, a clinician can grasp the pair of handles 36, 38 with a single hand 62 and lift the infant patient 60 for transport and movement.

In the embodiment depicted in FIG. 6, the warming device 32 may be inserted into the pocket 30 at either proximate or distal end 40, 42 to provide thermal support to the infant patient 60. The infant patient 60 is placed on the interior side 20 of the center section 12. First and second side sections 14, 16 can be brought towards one another to surround the infant patient 60. Handles 36, 38 can be brought in proximity of one another for grasping with a single hand for transport and movement of the infant patient 60.

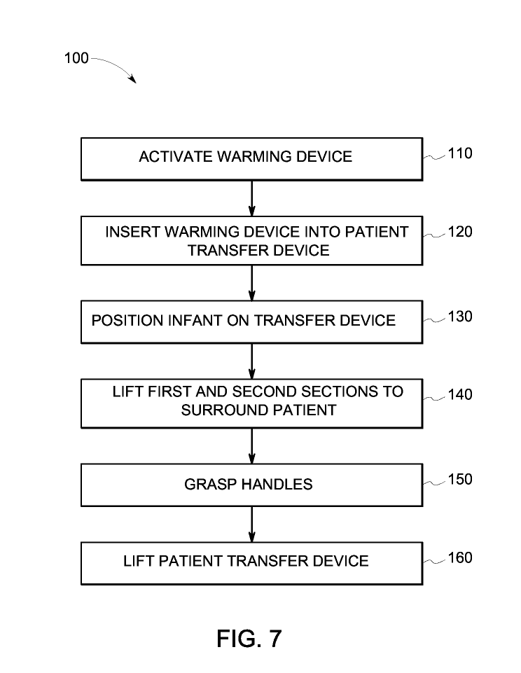

Referring back to FIGS. 1-6, the method 100 of utilizing the patient transfer device 10 of the present disclosure will now be described in connection with FIG. 7. Although the present method is being described as one preferred method of utilizing the patient transfer device 10, it should be understood that the patient transfer device 10 could be utilized in different ways for thermal support depending on the clinician requirement and the desired amount of movement necessary for the patient.

The method 100 may include a step 110 comprising activating the warming device 32. In one embodiment, the warming device 32 is an exothermic chemical reaction pouch. Activating this embodiment of warming device 32 may comprise physically bending, cracking or shaking the pouch to mix the components and begin an exothermic, heat producing reaction. In another embodiment, the warming device 32 is a phase change material. Activating this embodiment may comprise heating or melting the material to start a slow release of heat for a time period until the liquid has solidified, or changed phase. In yet another embodiment, the warming device 32 is a battery-operated electrical heating system device that produces thermal support when desired by activating or turning the device on.

The method 100 may also include a step 120 comprising inserting the warming device 32 into the patient transfer device 10. In one embodiment, the step 120 comprises coupling the warming device 32 to the patient transfer device 10 via the pocket 30 as described above with respect to FIG. 2. In another embodiment, the warming device 32 may be removably inserted into the pocket 30 at either the proximal end 40 or the distal end 42 as described above with respect to FIGS. 4 and 5.

The method 100 may also include a step 130 comprising positioning the infant patient 60 on the patient transfer device 10. In one embodiment, the patient transfer device 10 may be positioned within an incubator or patient bed before the infant patient 60 is placed within the incubator or bed. It is also contemplated that the patient transfer device 10 could be placed in an incubator in situations in which the infant patient 60 will be moved frequently by the clinician. The step 130 comprises placing the infant patient 60 on the center section 12 of the interior side 20 of the patient transfer device 10.

The method 100 may also include a step 140 comprising lifting the first and second side sections 14, 16 to surround the infant patient 60. In some embodiments, as the first and second side sections 14, 16 are brought upward toward each other, the handles 36, 38 become positioned in proximity to one another.

The method 100 may also comprise a step 150 of grasping the handles 36, 38. Once handles 36, 38 are positioned in proximity to one another, the handles 36, 38 can be grasped by a single hand 62 of the clinician as shown in FIGS. 3 and 6.

The method 100 may also comprise a step 160 comprising lifting the patient transfer device 10. Similarly to step 150, step 160 may be carried out with a single hand 62 of the caregiver, although the use of two hands is also within the scope of this disclosure.

As described herein, the patient transfer device 10 allows a clinician or caregiver to maneuver an infant patient 60 in a contained, physically and thermally supportive environment. The caregiver can smoothly use a single hand to lift and transfer the infant patient 60 to family, transport devices or medical beds without introducing negative touch. This may decrease or eliminate the consequences of negative touch which include deterioration of vital signs, inadvertent disconnections of medical devices or handling mishaps such as patient drops. The patient transfer device 10 also provides a tool for thermal support required by newborns immediately after birth to assist convective, evaporative, conductive and radiant heat loss. This may reduce the likelihood of hypothermia, a leading cause of neonatal morbidities and mortalities.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A transfer device for warming an infant patient during movement of the patient, comprising:
a center section positionable beneath the patient, the center section comprising a warming device;
a first side section connected to the center section and including a first handle; and
a second side section connected to the center section and including a second handle;
wherein the first and the second side sections are movable toward each other such that the first and second handles are located proximate to each other and the first and second side sections surround the patient.

2. The transfer device of claim 1, wherein the warming device is removably positioned within the center section.

3. The transfer device of claim 1, wherein the warming device is coupled to an exterior side of transfer device.

4. The transfer device of claim 1, wherein the warming device comprises an exothermic chemical reaction pouch.

5. The transfer device of claim 1, wherein the warming device comprises a phase change material.

6. The transfer device of claim 1, wherein the warming device is a battery-operated electrical heating device.

7. The transfer device of claim 1, wherein the center section comprises a pocket, a portion of which is fixedly attached to the center section.

8. The transfer device of claim 1, wherein the center section comprises a pocket that is coupled to the center section.

9. A transfer device for warming an infant patient during movement of the patient, comprising:
a center section positionable beneath the patient;
a warming device removably positioned within the center section;
a first side section connected to the center section;
a second side section connected to the center section, wherein the first and second side sections are sized to surround the infant patient when the first and second side sections are brought towards each other.

10. The transfer device of claim 9, wherein the warming device is an exothermic chemical reaction pouch.

11. The transfer device of claim 9, wherein the warming device is a phase change material.

12. The transfer device of claim 9, wherein the warming device is a battery-operated electrical heating device.

13. A method of warming an infant patient during movement of the patient, comprising:
activating a warming device;
inserting the warming device into a center section of a patient transfer device;
positioning the transfer device beneath the patient, the transfer device comprising a first side section and a second side section, the first side section and the second side section connected to the center section, wherein the infant is positioned on the center section; and
lifting first and second sections to surround the infant patient.

14. The method of claim 13, further comprising:
grasping a first handle connected to the first side section and a second handle connected to the second side section; and
lifting the transfer device and the infant patient.

15. The method of claim 13, further comprising:
grasping a first handle connected to the center section and a second handle connected to the center section; and
lifting the transfer device and the infant patient.

16. The method of claim 13, wherein the warming device comprises an exothermic chemical reaction pouch.

17. The method of claim 13, wherein the warming device comprises a phase change material.

18. The method of claim 13, wherein the warming device is battery-operated electrical heating device.
